(19) United States
(12) Patent Application Publication
(10) Pub. No.: US 2008/0243179 A1
(43) Pub. Date: Oct. 2, 2008

(54) GROOMING DEVICE FOR EYEBROWS

(76) Inventor: Eyal Ziv, Tel Aviv (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 11/692,386

(22) Filed: Mar. 28, 2007

(51) Int. Cl. A61B 17/50

(52) U.S. Cl. 606/211

(57) ABSTRACT

Presented herein is a grooming device for the removal of hair and for eyebrow care and maintenance, said device comprising a pair of tweezers and a brush head disposed on said tweezers. Some embodiments of the grooming device of the present invention further comprise a protective storage case comprising a receiving member and a cover member that has a mirror disposed thereon. Some embodiments of the grooming device of the present invention further comprise a light assembly disposed on the tweezers.

### FIELD OF INVENTION

This invention relates generally to grooming devices, and especially to devices used to maintain hair.

### BACKGROUND OF THE INVENTION

Personal grooming routines often include removing unwanted hair, a common problem for many people. For example, many women remove individual hairs in order to reshape their eyebrows.

It is important, when grooming the eyebrows, that the hairs be removed accurately. This generally involves three tools.

A mirror enables one to observe the eyebrow. A small eyebrow brush smoothes out the eyebrows to help identify which eyebrow hairs need to be removed. A pair of cosmetic tweezers is used to pluck and remove the identified hairs. The brow brush is used throughout the process to re-smooth the eyebrows to ensure that the correct hairs are removed and that the desired brow shape is being achieved.

During this process, the user must either hold the brush in one hand and the tweezers in the other or continuously put one tool down and pick up the other. The inconvenience is compounded if one does not have a freestanding mirror and the mirror must be held in one hand and the tweezers or brush in the other.

Currently, there is no single grooming device that comprises a pair of tweezers and a brow brush.

### SUMMARY OF THE INVENTION

Presented herein is a grooming device for the removal of hair and especially for the shaping, care, and maintenance of facial hair such as eyebrows.

A key feature of the present invention is a brush head that is disposed on a pair of tweezers. Some embodiments of the grooming device of the present invention are further equipped with a light assembly. Additionally, a storage case may be provided for storing any embodiment of the present invention.

This novel combination offers a number of advantages, including:
The grooming device of the present invention enables the user to both pluck unwanted eyebrow hairs and brush or shape the eyebrow with a single tool.
The grooming device of the present invention requires the use of only one hand, offering greater convenience to the user.
The compact design of the grooming device makes the grooming device especially suitable for travel.

Accordingly, a grooming device of the present invention for the removal of hair and for eyebrow care and maintenance is provided, said device comprising a pair of tweezers and a brush head disposed on said tweezers.

Additionally, the brush head may be attached to the tweezers by at least one suitable fastener.

Additionally, the brush head may be rotatably coupled to the tweezers, thereby enabling the brush head to be stowed between the legs of the tweezers.

Additionally, the grooming device may be manufactured as a single piece.

Additionally, the brush head may be disposed on the tweezers substantially vertically or at an angle.

The grooming device may further comprise a protective storage case. Such a case may comprise a receiving member adapted to receive the grooming device, a cover member that conforms to the shape of at least one side of the receiving member and covers an open end of the receiving member, and a mirror disposed on a surface of the cover member.

The grooming device may further comprise a light assembly disposed on the tweezers.

Additionally, the light assembly may be disposed between the legs of the tweezers.

Additionally, the brush head may be removable to provide access to the light assembly.

### BRIEF DESCRIPTION OF THE DRAWINGS

The subject matter regarded as the invention will become more clearly understood in light of the ensuing description of embodiments herein, given by way of example and for purposes of illustrative discussion of the present invention only, with reference to the accompanying drawings, wherein

FIG. 1 is a perspective view of grooming device for eyebrow care and maintenance, according to an embodiment of the present invention;

FIG. 2 is a perspective view of grooming device for eyebrow care and maintenance, according to another embodiment of the present invention;

FIG. 3A is a perspective view of a closed storage case for a grooming device, according to some embodiments of the present invention; and

FIG. 3B is a perspective view of the storage case of FIG. 3A, opened.

The drawings together with the description make apparent to those skilled in the art how the invention may be embodied in practice.

No attempt is made to show structural details of the invention in more detail than is necessary for a fundamental understanding of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DESCRIPTION OF SOME EMBODIMENTS OF THE INVENTION

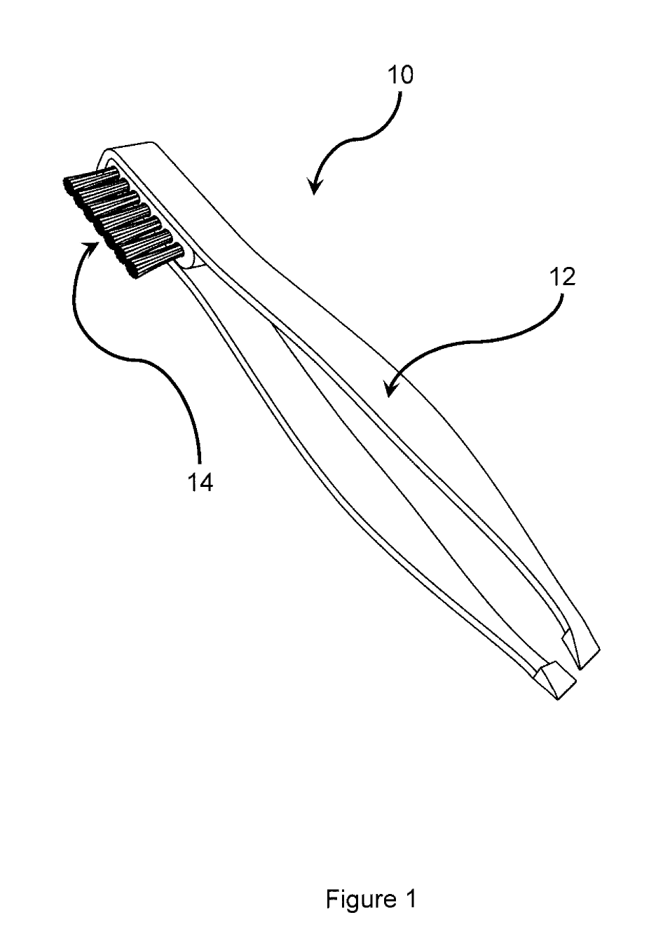

Referring now in detail to FIGS. 1, 2, 3A, and 3B, embodiments of a grooming device 10 according to the present invention comprise a pair of tweezers 12 and a brush head 14 disposed at the joined end of said tweezers 12.

An embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments” or “other embodiments” means that a particular feature, structure, or characteristic described in connection with the embodiments is included in at least one embodiment, but not necessarily all embodiments, of the inventions.

It is understood that the phraseology and terminology employed herein is not to be construed as limiting and is for descriptive purpose only.

The principles and uses of the teachings of the present invention may be better understood with reference to the accompanying description, figures, and examples.

It is to be understood that the details set forth herein do not construe a limitation to an application of the invention. Furthermore, it is to be understood that the invention can be carried out or practiced in various ways and that the invention can be implemented in embodiments other than the ones outlined in the description below.

It is to be understood that the terms “including”, “comprising”, “consisting” and grammatical variants thereof do not preclude the addition of one or more components, features, steps, integers or groups thereof and that the terms are not to be construed as specifying components, features, steps or integers.

The phrase “consisting essentially of”, and grammatical variants thereof, when used herein is not to be construed as excluding additional components, steps, features, integers or groups thereof but rather that the additional features, integers, steps, components or groups thereof do not materially alter the basic and novel characteristics of the claimed composition, device or method.

If the specification or claims refer to “an additional” element, that does not preclude there being more than one of the additional element.

It is to be understood that where the claims or specification refer to “a” or “an” element, such reference is not to be construed as there being only one of that element.

It is to be understood that where the specification states that a component, feature, structure, or characteristic “may”, “might”, “can” or “could” be included, that particular component, feature structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” refers to manners, means, techniques and procedures for accomplishing a given task including, but is not limited to those manners, means, techniques and procedures either known to, or readily developed from known manners, means, techniques and procedures by practitioners of the art to which the invention belongs.

The descriptions, examples, methods, and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention can be implemented in the testing or practice with methods and materials equivalent or similar to those described herein.

The terms “bottom”, “below”, “top” and “above” as used herein do not necessarily indicate that a “bottom” component is below a “top” component or that a component that is “below” is indeed “below” another component or that a component that is “above” is indeed “above” another component. As such, directions, components or both may be flipped, rotated, moved in space, placed in a diagonal orientation or position, placed horizontally or vertically, or similarly modified. Accordingly, it will be appreciated that the terms “bottom”, “below”, “top” and “above” may be used herein for exemplary purposes only, to illustrate the relative positioning or placement of certain components, to indicate a first and a second component or to do both.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

According to embodiments of the present invention, tweezers 12 may be any type of pincers suitable for plucking hairs, singly or a few at a time.

According to embodiments of the present invention, the blades, or tips, of tweezers 12 may be, inter alia square, slanted, angled, rounded, or pointed or any other shape suitable for plucking hairs. Additionally, according to embodiments, the legs, also referred to as grips, handles or prongs, of tweezers 12 may be, inter alia, regular or wide-grip.

It is understood that tweezers 12 may have other designs and shapes without departing from the scope of the invention.

According to embodiments of the present invention, a brush head 14 is disposed on tweezers 12 at the joined or fulcrum end of tweezers 12.

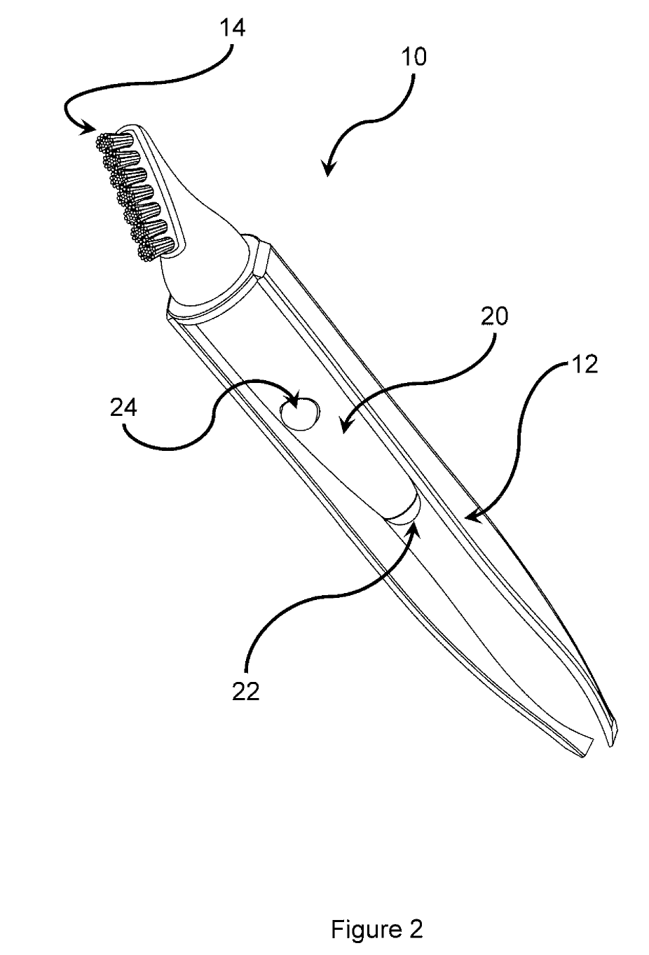

According to some embodiments, brush head 14 may be located between the legs of tweezers 12, as shown in FIG. 1. According to some other embodiments, brush head 14 may be disposed on the end of tweezers 12, as seen in FIG. 2.

According to some embodiments of the present invention, brush head 14 may be disposed on tweezers 12 substantially parallel to tweezers 12, as seen in FIG. 1. According to some other embodiments, brush head 14 may be disposed on tweezers 12 at an angle, as seen in FIG. 2.

It is understood that brush head 14 may also be placed elsewhere on tweezers 12 and at different angles without departing from the scope of the present invention.

The bristles of brush head 14 may be made of any suitable material or combination of materials, including natural and synthetic material.

According to some embodiments of the present invention, brush head 14 may be permanently attached to tweezers 12 by at least one suitable fastener. The fastener may be at least one of, inter alia, glue, a weld, a screw, a groove, a track, paired threads, a snap, and interconnecting notches. It is understood that other fasteners and fastening methods may be used without departing from the scope of the invention.

According to some other embodiments of the present invention, brush head 14 may be removably attached to the tweezers 12 by at least one suitable fastener. The fastener may be at least one of, inter alia, one or more grooves on tweezers 12 and brush head 14, one or more tracks on tweezers 12 and brush head 14, paired threads on tweezers 12 and brush head 14, a snap and interconnecting notches on tweezers 12 and brush head 14. It is understood that other fasteners and fastening methods may be used without departing from the scope of the invention.

Additionally, according to some embodiments of the present invention, the fastener may rotatably couple brush head 14 to tweezers 12. Such a fastener would enable brush head 14 to, for example, be conveniently stowed between the legs of tweezers 12.

According to yet other embodiments of the present invention, tweezers 12 and brush head 14 of grooming device 10 may be manufactured as a single piece.

According to some embodiments of the present invention, a grooming device 10 may further comprise a light assembly 20 disposed on tweezers 12. Light assembly 20 may be disposed, for example, between the legs of tweezers 12. Light assembly 20 may, for example, contain a light emitting diode type light 22 that may be operated by, for example, a switch 24. Such an embodiment is described in FIG. 2.

According to such embodiments, light assembly 20 may, for example, project a light beam to an area that encompasses, for example, the tips of tweezers 12 and the immediate area to be tweezed.

It should be understood that a light assembly 20 is not necessary to use the present invention, and is merely an optional feature.

According to some embodiments of the present invention, removing brush head 14 enables access to light assembly 20, allowing the user to, for example, change a battery contained therein.

According to some embodiments of the present invention, brush head 14 may be rotatably coupled to tweezers 12 thereby enabling the brush head 14 to be stowed between the legs of tweezers 12, alongside light assembly 20.

According to some embodiments of the present invention, grooming device 10 further comprises a protective storage ease 30 within which grooming device 10 may be stored.

It should be understood that a protective storage case 30 is not necessary to use the present invention, and is merely an optional feature.

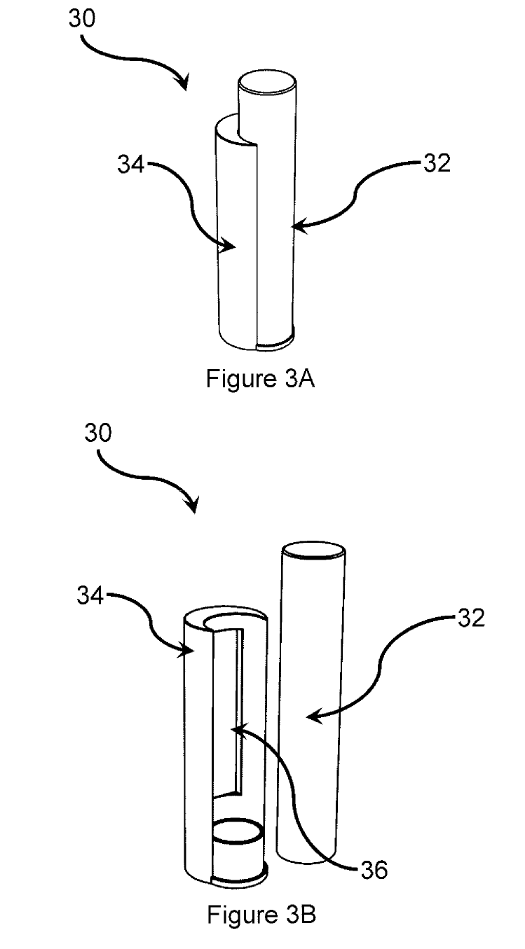

According to some embodiments of the present invention, a storage case 30 comprises a receiving member 32 and a cover member 34, as described in FIGS. 3A and 3B.

According to such embodiments, receiving member 32 is substantially cylindrical in shape and is adapted to receive grooming device 10 therein and cover member 34 has a base that covers an open end of receiving member 32 and an extended section that conforms to the shape of at least one exterior surface of receiving member 32. This configuration enables cover member 34 to slide over and close receiving member 32 and securely contain grooming device 10 therein.

According to some embodiments of the present invention, storage case 30 may further comprise a mirror 36. Mirror 36 may be magnifying or non-magnifying. Mirror 36 may be disposed, for example, on an interior surface of cover member 34, as shown in FIG. 3B. It should also be understood that a mirror 36 is not necessary to use the grooming device, and is merely an optional feature.

It is furthermore understood that other constructions, designs, and configurations could be used for protective storage case 30 without departing from the scope of the invention.

While the invention has been described with respect to a limited number of embodiments, these should not be construed as limitations on the scope of the invention, but rather as exemplifications of some of the embodiments. Those skilled in the art will envision other possible variations, modifications, and applications that are also within the scope of the invention. Accordingly, the scope of the invention should not be limited by what has thus far been described, but by the appended claims and their legal equivalents. Therefore, it is to be understood that alternatives, modifications, and variations of the present invention are to be construed as being within the scope and spirit of the appended claims.

What is claimed is:

1. A grooming device for the removal of hair and for eyebrow care and maintenance, said device comprising
a pair of tweezers and
a brush head disposed on said tweezers.

2. The grooming device of claim 1, wherein the brush head is attached to the tweezers by at least one suitable fastener.

3. The grooming device of claim 2, wherein the fastener is at least one of
glue,
weld,
screw,
groove,
track,
paired threads,
snap, and
interconnecting notches.

4. The grooming device of claim 3, wherein the fastener rotatably couples the brush head to the tweezers.

5. The grooming device of claim 4, wherein the fastener enables the brush head to be stowed between the legs of the tweezers.

6. The grooming device of claim 1, wherein the grooming device is manufactured as a single piece.

7. The grooming device of claim 1, wherein the brush head is disposed on the tweezers at an angle.

8. The grooming device of claim 1, further comprising a protective storage case.

9. The grooming device of claim 8, wherein the storage case comprises
a receiving member adapted to receive the grooming device,
a cover member that conforms to the shape of at least one side of the receiving member and covers an open end of the receiving member, and
a mirror disposed on the inner surface of the cover member.

10. The grooming device of claim 1, further comprising a light assembly disposed on the tweezers.

11. The grooming device of claim 10, wherein the light assembly is disposed between the legs of the tweezers.

12. The grooming device of claim 10, wherein the brush head is rotatably coupled to the tweezers thereby enabling the brush head to be stowed between the legs of the tweezers.

13. The grooming device of claim 10 wherein the brush head is removably attached to the tweezers by at least one suitable fastener.

14. The grooming device of claim 13, wherein the fastener is at least one of:
paired threads,
snap and
interconnecting notches

15. The grooming device of claim 10, wherein the brush head is removable to provide access to the light assembly.
